(19) United States
(12) Patent Application Publication
(10) Pub. No.: US 2013/0245606 A1
(43) Pub. Date: Sep. 19, 2013

(54) HYDROGEL BASED OCCLUSION SYSTEMS

(71) Applicants: UNIVERSITY COLLEGE CORK, NATIONAL UNIVERSITY OF IRELAND, CORK, (US); INTERUNIVERSITAIR MICRO-ELECTRONICA CENTRUM VZW, (US); KATHOLIEKE UNVIVERSITEIT LEUVEN - KU LEUVEN RESEARCH & DEVELOPMENT, (US); UNIVERSITEIT GENT, Gent (BE); RAMOT AT TEL-AVIV UNIVERSITY, LTD., Tel Aviv (IL); TECHNISCHE UNIVERSITEIT DELFT, Delft (NL)

(72) Inventors: Frank Albert STAM, Aherla (IE); Nathan JACKSON, Cork (IE); Peter DUBRUEL, EINE (BE); Kehinde ADESANYA, Ghent (BE); Anika EMBRECHTS, Pijnacker (NL); Eduardo MENDES, JR., Delft (NL); Hurcules Pereira NEVES, Hamme-Mille (BE); Paul HERIJGERS, Kampenhout (BE); Peter VERBRUGGHE, Rotselaar (BE); Yosi SHACHAM, Zichron Yaacov (IL); Leeya ENGEL, Tel Aviv (IL); Viacheslav KRYLOV, Tel Aviv (IL)

(73) Assignees: UNIVERSITY COLLEGE CORK, NATIONAL UNIVERSITY OF IRELAND, CORK, Cork (IE); UNIVERSITEIT GENT, Gent (BE); TECHNISCHE UNIVERSITEIT DELFT, Delft (NL); RAMOT AT TEL-AVIV UNIVERSITY, LTD., Tel Aviv (IL); KATHOLIEKE UNVIVERSITEIT LEUVEN - KU LEUVEN RESEARCH & DEVELOPMENT, Leuven (BE); INTERUNIVERSITAIR MICRO-ELECTRONICA CENTRUM VZW, Leuven (BE)

(21) Appl. No.: 13/832,069

(22) Filed: Mar. 15, 2013

Related U.S. Application Data
(60) Provisional application No. 61/611,380, filed on Mar. 15, 2012.

(51) Int. Cl. A61B 17/12

(52) U.S. Cl. CPC A61B 17/12181
USPC 604/507; 604/57; 264/41

(57) ABSTRACT

A hydrogel based occlusion system, a method for occluding vessels, appendages or aneurysms, and a method for hydrogel synthesis are disclosed. The hydrogel based occlusion system includes a hydrogel having a shrunken and a swollen state and a delivery tool configured to deliver the hydrogel to a target occlusion location. The hydrogel is configured to permanently occlude the target occlusion location in the swollen state. The hydrogel may be an electro-activated hydrogel (EAH) which could be electro-activated with a delivery system to control the degree of swelling/shrinking.

### FIELD OF THE INVENTION

The invention relates generally to hydrogel based occlusion systems, and in particular to systems and methods for occluding cardiovascular and cerebral arteries and aneurysm.

### BACKGROUND OF THE INVENTION

During the last decade there has been a significant progress in the development of smart materials that target biological and bio-medical applications. Among those materials, polymer based hydrogels were investigated as they can resemble normal physiological conditions due to their high water content. Water based gels may be used for controlled drug delivery and release systems for example.

In addition, hydrogels are widely used in tissue engineering. Apart from the above mentioned bio-medical applications, hydrogels may be used as precise biosensors and diagnostic devices.

Polyelectrolyte hydrogels, which are polymer hydrogels with charged groups incorporated into their macromolecular network, are capable of volumetric and/or mechanical changes in the presence of external stimuli like electric field, pH or specific salt solution due to their chemical structure.

In case of electro-activated hydrogels, control over swelling, shrinking and bending behavior in response to external fields may be used to achieve direct conversion of electrical energy into mechanical energy. This makes polyelectrolyte hydrogels good candidates for biomedical applications.

Hydrogels may be used for the treatment of cardiovascular diseases requiring vessel occlusion, device sealing or cavity filling, taking advantage of the hydrogels ability to change form and shape due to the presence of external stimuli like electric field or environmental parameter changes like temperature and pH. However, there are only a few examples of implanted medical devices that use hydrogels today and these applications are based typically on coils, beads or shape memory materials, only coated with hydrogels and where the hydrogels are not electro-activated.

An aortic aneurysm is a general term for any swelling (dilation or aneurysm of the aorta to greater than 1.5 times normal, usually representing an underlying weakness in the wall of the aorta at that location. While the stretched vessel may occasionally cause discomfort, a greater concern is the risk of rupture, which causes severe pain; massive internal hemorrhage and, without prompt treatment, death occurs rapidly.

A cerebral or brain aneurysm is a cerebrovascular disease disorder in which weakness in the wall of a brain Artery or Vein causes a localized vasodilation or ballooning of the blood vessel. If an aneurysm ruptures, it leaks blood into the space around the brain. This is called a “subarachnoid hemorrhage.” Depending on the amount of blood, it can produce a sudden severe headache that can last from several hours to days, nausea and vomiting, drowsiness and/or coma. The ruptured aneurism (hemorrhage) may also damage the brain directly, usually from bleeding into the brain itself. This is called a “hemorrhagic stroke.” This can lead to weakness or paralysis of an arm or leg, trouble speaking or understanding language, vision problems, seizures.

It would be highly advantageous to develop hydrogels based occlusion systems that may be used to occlude blood vessels, aortic and cerebral aneurysms

### SUMMARY OF THE INVENTION

According to certain embodiments there is provided a hydrogel based occlusion system, a method for occluding vessels or aneurysms, and a method for hydrogel synthesis.

The hydrogel based occlusion system includes a hydrogel having a shrunken and a swollen state and a delivery tool configured to deliver the hydrogel to a target occlusion location. The hydrogel is configured to occlude permanently the target occlusion location in the swollen state.

In one embodiment, the hydrogel based occlusion system comprising further an external device used to shrink the hydrogel before deployment using the delivery tool.

In another embodiment, the hydrogel has a solid symmetrical shape around its longitudinal axis, wherein the delivery tool is configured to deliver the hydrogel to the target occlusion location using a mechanical assisted detachment system.

In one embodiment, the hydrogel is generally cylindrically shaped and has a central hole along its cylindrical axis, wherein the delivery tool is configured to deliver the hydrogel to the target occlusion location using a guide wire configured to thread through the central hole, and wherein the central hole is configured to be filled by the hydrogel in the swollen state after the guide wire is removed, thereby occluding the target location.

In another embodiment, the hydrogel comprises an electro-activated hydrogel (EAH) and wherein the occlusion system comprises further a stimulator for applying electrical stimulations to and EAH to maintain or change the state of expansion.

In one embodiment, the hydrogel shrunken state is a substantially dried state and the swollen state is a wet state in equilibrium in fluid contact with blood.

In another embodiment, the target occlusion location is selected from the group consisting of: a blood vessel wherein the occlusion is performed to block blood flow through the vessel; a hepatic artery wherein the occlusion is performed to stop blood flow to a tumor; aortic aneurysm wherein the occlusion is performed to block aortic aneurysm growth and rupture; and cerebral aneurysm wherein the occlusion is performed to block cerebral aneurysm growth and rupture.

In one embodiment, the stimulator is configured to apply electrical stimulations to the EAH to bring it to its shrunken state before deployment, wherein the EAH is deployed in the target occlusion location in its shrunken state and wherein the EAH is configured to expand to its swollen state when-electrical stimulation is not applied thereon.

In another embodiment, the delivery tool comprises an introducer sheath, a dilator, an insertion guide, a pusher, a guide wire and a hydrogel protrusion set, wherein the guide wire is inserted first to the target occlusion location through the introducer sheath, wherein the hydrogel is placed in the hydrogel protrusion set in its shrunken state, wherein the pusher is configured to push the hydrogel protrusion set to the target occlusion location on the guide wire and to push the hydrogel out of the hydrogel protrusion set at the target occlusion location after it expands to the swollen state, thereby occluding the target location.

In one embodiment, the guide wire comprises further a J shape distal end configured to hold the hydrogel in the target occlusion location until it expands to the swollen state.

In another embodiment, the hydrogel protrusion set comprises further a balloon, the balloon configured to prevent blood flow around the hydrogel at the target location during expansion of the hydrogel to its swollen state.

In one embodiment, the hydrogel protrusion set comprises further at least two electrodes configured to apply electrical stimulation to the EAH.

In another embodiment, the first electrode of the at least two electrodes is a central electrode located in the EAH central hole, and a second electrode is an outer electrode located at the hydrogel protrusion set perimeter.

In one embodiment, the delivery tool comprises further a plurality of interdigitated electrodes, wherein the plurality of interdigitated electrodes is arranged to uniformly shrink the hydrogel either externally before deployment or internally in the patient body.

In another embodiment, the hydrogel includes biomimetic linking molecules that target vascular endothelial receptors, and wherein a drug is incorporated in the interior of the hydrogel in order to enhance ligand-receptor interaction strengthening chemical adhering between the hydrogel and the vessel wall.

In one independent embodiment, an occlusion method is disclosed. The method includes: providing a hydrogel with a shrunken state and a swollen state and a delivery tool; delivering the hydrogel in its shrunken state to an occlusion target location in a patient body; detaching the hydrogel from the delivery tool after it expands to its swollen state, thereby permanently occluding the target occlusion location; and removing the delivery tool from the patient body.

In another embodiment, the provided hydrogel is an electro-activated hydrogel (EAH), and wherein the method further comprising inserting the provided EAH into the delivery tool and stimulating the EAH to a shrunken state either externally before deployment or internally in the patient body.

In one embodiment, stimulating the provided EAH to a shrunken state is performed before inserting the EAH into the delivery tool.

In another embodiment, the method further comprising during the delivery of the EAH, stimulating the EAH to control the swelling rate of the EAH during the delivering. In another independent embodiment, a hydrogel synthesis method is disclosed. The method comprises the steps of: (a) flushing de-ionized distilled water with nitrogen gas for about 30 minutes; (b) dissolving 8-15% per solution of water of pluronic-BMA powder (PH127) in 24-25% methacrylic acid liquid; (c) adding the de-ionized distilled water to the pluronic-BMA solution up to 85% of the total weight; (d) preparing 1-10 M ammonium persulphate (APS) solution of the de-ionized distilled water flushed in nitrogen gas; (e) preparing 1-10 M TEMED solution of the de-ionized distilled water flushed in argon gas; (f) mixing at room temperature and pluronic-BMA, APS and TEMED solutions, thereby forming an EAH solution and adding. 5-10% porogens in order to generate porosity in the EAH solution; (g) cooling the EAH solution on ice for several hours; (h) stirring the cooled EAH solution for several minutes; (i) cooling the stirred EAH solution for 8-12 hours at about 4 degrees Celsius; (j) adding 7.5% APS and 7.5% TEMED to the EAH solution; (k) pouring the cooled EAH mixture into a mold and leaving the EAH solution to settle for about 1 hour at about 4 degrees Celsius in the mold; (1) curing the EAH mixture in a 37° C. water bath for about 3 hours; and (m) removing the porogens by a leaching out method using excessive solvent.

Additional features and advantages of the invention will become apparent from the following drawings and description.

### BRIEF DESCRIPTION OF THE DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 illustrates a hydrogel based occlusion delivery system including a hemostasis valve, introducer sheath and dilator, according to certain embodiments;

FIGS. 2A-2D illustrate a guide insertion, dilator extraction and the hydrogel protrusion set insertion over a guidewire, according to certain embodiments;

FIGS. 3A-3D illustrate a hydrogel protrusion set, catheter extraction, the gel expansion and detachment, according to certain embodiments;

FIGS. 4A-4D illustrate a hydrogel protrusion set including a balloon, according to certain embodiments;

FIG. 5A illustrates a hydrogel protrusion set that includes electro-activating electrodes, according to certain embodiments;

FIG. 5B illustrates an interdigitated electrode configuration, according to certain embodiments;

FIG. 6 illustrates a hydrogel protrusion set that includes a pusher with teeth , according to certain embodiments;

FIG. 7 illustrates gel synthesis, according to certain embodiments;

FIG. 8 illustrates a method of occlusion based on hydrogel, according to certain embodiments; and

FIG. 9 illustrates a method of occlusion based on electro-activated hydrogel, according to certain embodiments.

### DESCRIPTION OF THE PREFERRED EMBODIMENTS

A hydrogel based occlusion system, a method for occluding vessels or aneurysms, and a method for hydrogel synthesis are disclosed. The occlusion system includes a hydrogel having a shrunken and a swollen state and a delivery tool configured to deliver the hydrogel to a target occlusion location. The hydrogel is configured to occlude permanently the target occlusion location in its second state.

Optionally, the hydrogel has a central hole along its cylindrical axis enabling delivering the hydrogel to the target occlusion location over a guidewire configured to thread through the hydrogel central hole. The central hole is configured to be filled by the hydrogel after it swells occluding the target location.

Optionally, the hydrogel is an electro-active hydrogel (EAH) and the occlusion system, and/or the occlusion delivery system, includes a stimulator for applying electrical stimulations onto the EAH to transform it from state to state.

Optionally, the hydrogel shrunken state is a substantially dried state and the swollen state is a wet state in equilibrium in fluid contact with blood.

Optionally, the hydrogel shrunken state is an electro-activated contracted state and the hydrogel swollen state is an expanded state due to swelling with no electro-actuation applied and due to fluid contact with the patient blood.

According to certain embodiments, the hydrogel based occlusion system may be used to occlude a blood vessel wherein the occlusion is performed to block blood flow through the vessel. The occlusion system may be used to occlude a hepatic artery wherein the occlusion is performed to stop blood flow to a tumor. The occlusion system may be used to occlude an aortic aneurysm wherein the occlusion is performed to block aortic aneurysm growth and rupture and cerebral aneurysm wherein the occlusion is performed to block cerebral aneurysm growth and rupture.

According to certain embodiments, the synthesized hydrogel disclosed is non-toxic, non-hemolytic, does not elicit any undesirable local or systematic effect, and is bio-compatible (tested according to biocompatibility guidelines such as ASTM F756 and ISO-10993).

### Hydrogel Based Occlusion System

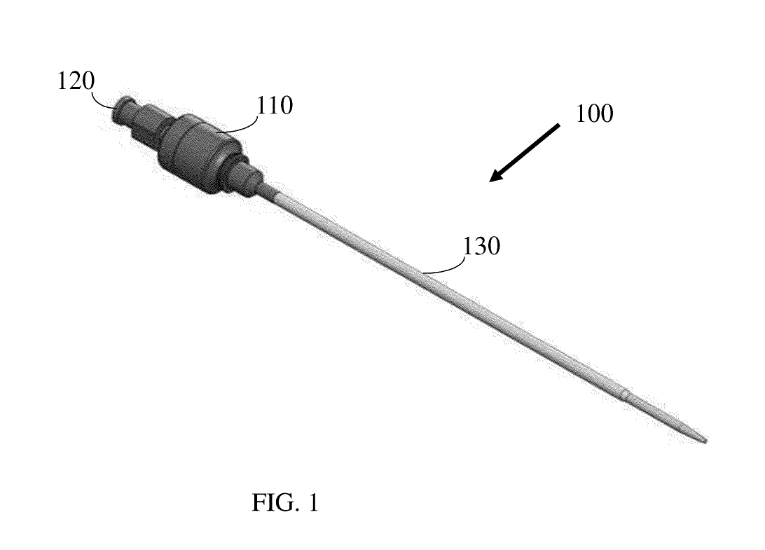

FIG. 1 illustrates a hydrogel based occlusion delivery system 100 that includes a hemostasis valve 110, introducer sheath 130 and dilator 120, according to certain embodiments. Occlusion delivery system 100 includes a basic pusher catheter and hydrogel protrusion set described further below. The hemostasis valve 110 has a wide entrance hole 120 for the insertion of dilator 120 in its proximal end. The introducer sheath 130 and dilator 120 are standard commercially available sheath and dilator typically 40-50 cm long and having a 7 French diameter. Other introducer sheath lengths and diameters (between 2 and 30 Frenches) may be used in order to match various sizes of vessels, or aneurysms to be occluded.

Optionally, the hydrogel occlusion system may include an imaging means used to validate the positioning of the hydrogel occlusion in the vessel or in the aneurysm wherein the delivery tool distal end may be made of an x-ray opaque material or the hydrogel may includes x-ray opaque material.

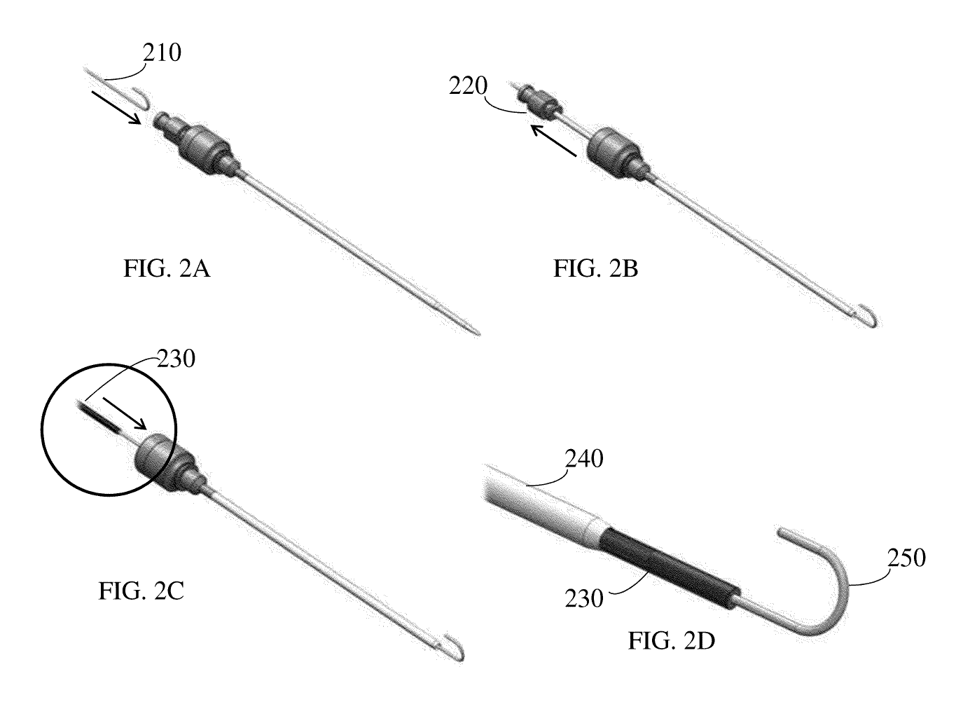

The step-by-step procedure for the delivery of the hydrogel occlusion is described in FIGS. 2-6 below. The procedure starts with a needle insertion into a blood vessel followed by insertion of the introducer sheath 130 distal end to the blood vessel.

FIGS. 2A-2D illustrate the guidewire insertion, dilator extraction and the hydrogel protrusion set insertion over a guidewire, according to certain embodiments. Guidewire 210 is inserted through the dilator to the blood vessel until it reaches the occlusion target location. Optionally, the guidewire has a J shape distal end 250 used to hold the hydrogel in the target location before the hydrogel swells and occlude the blood vessel. After the guidewire in inserted, dilator 220 is extracted and the hydrogel protrusion set 230 is inserted over the guidewire. The hydrogel protrusion set 230 is installed over the proximal end of guidewire 210 and pushed through an introducer sheath 240 while the guidewire 210 is held steady.

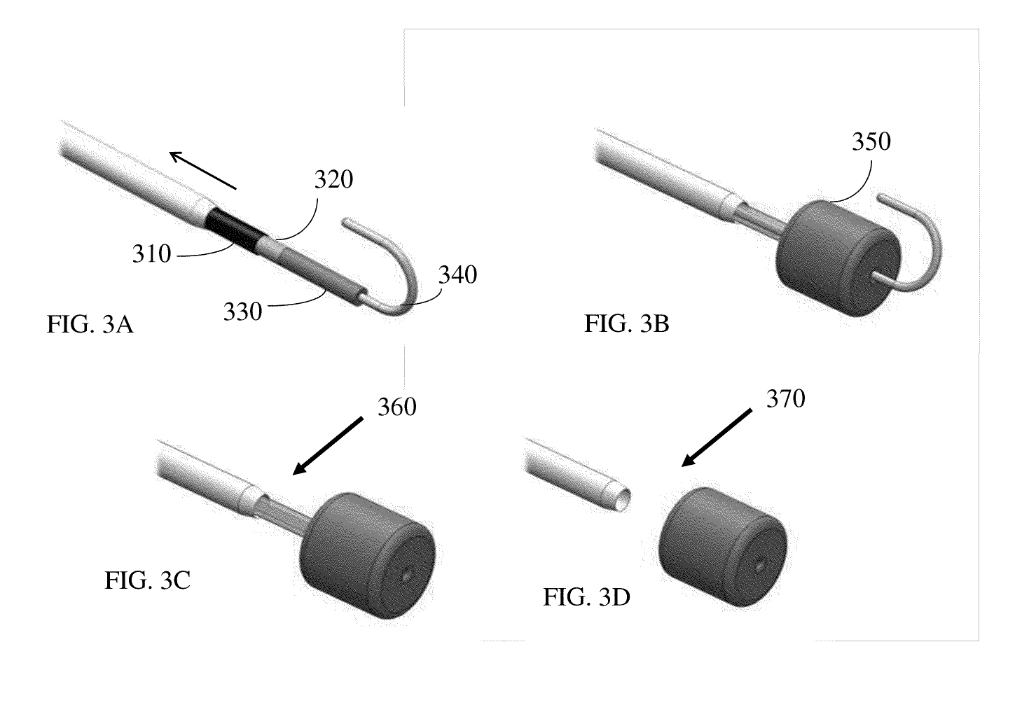

FIGS. 3A-3D illustrate a hydrogel protrusion set, the catheter extraction, and the gel expansion and detachment, according to certain embodiments. The hydrogel protrusion set includes a catheter 310 pusher 320 and hydrogel 330 in its shrunken state. The guidewire J shape distal end 340 is used to hold the hydrogel in the target location in the blood flow in its shrunken state before swelling.

Optionally, Hydrogel 330 has a solid symmetrical shape around its longitudinal axis. Hydrogel 330 may have a substantially cylindrical shape and a central hole along its cylindrical axis enabling delivering the hydrogel to the target occlusion location over a guidewire configured to thread through the hydrogel central hole.

The hydrogel swells to its swollen state 350 due to fluid contact with the patient blood and adheres to the vessel walls in its swollen state. The guidewire is extracted 360 and the hydrogel swelling fills the central hole occluding the blood vessel permanently. The pusher is extracted through the introducer sheath 370 and the hydrogel is detached from the delivery tool.

The hydrogel shrunken state may be a substantially dried state and the hydrogel swollen state may be a swollen state in equilibrium in fluid contact with blood.

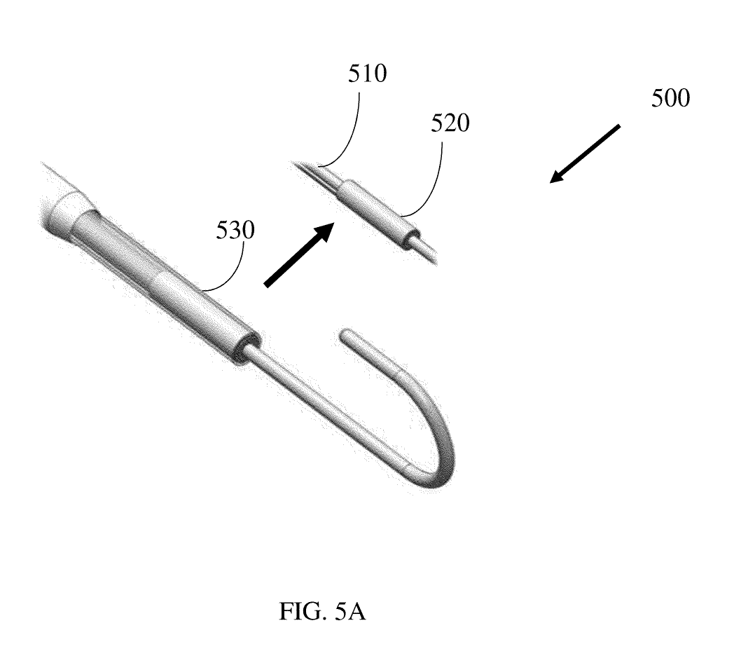

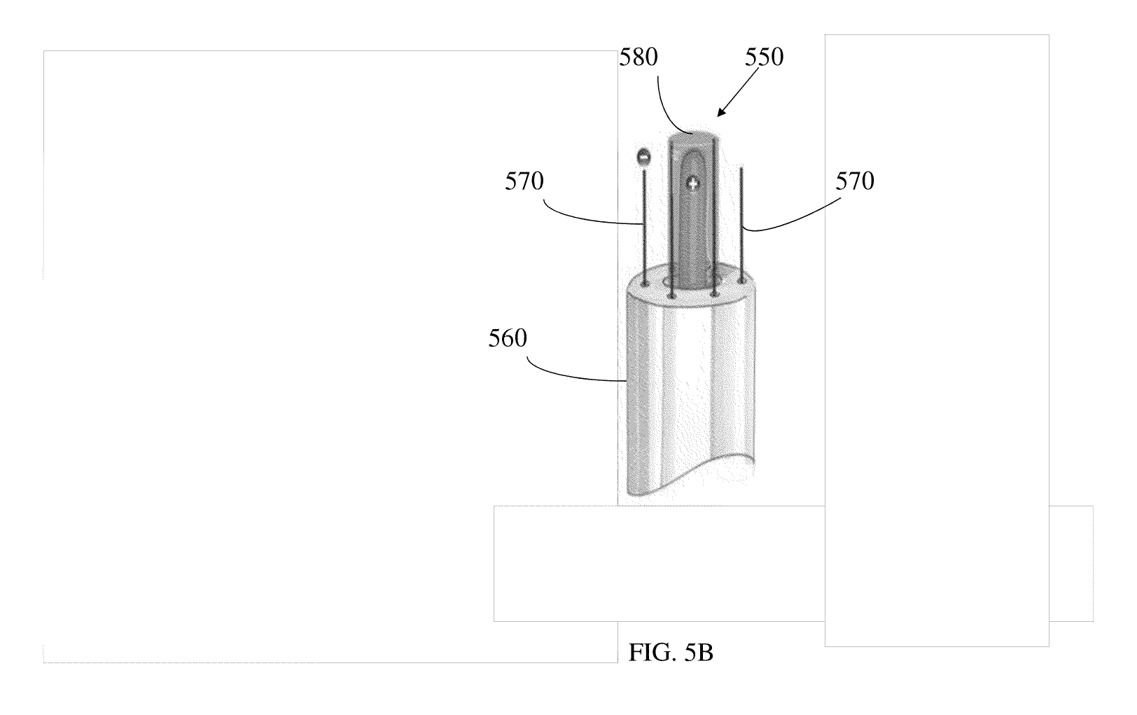

Optionally, the hydrogel shrunken state is an electro-activated contracted state and the hydrogel swollen state is an expanded state due to swelling with no electro-actuation applied and due to fluid contact with the patient blood as described further below regarding FIG. 5.

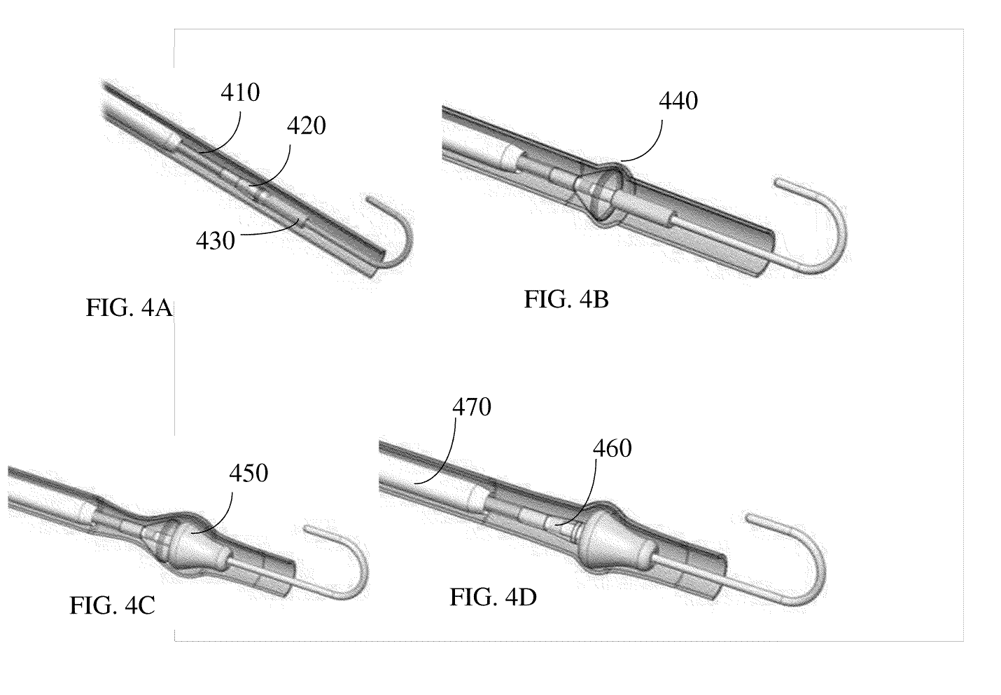

FIGS. 4A-4D illustrate the hydrogel protrusion set including a balloon, according to certain embodiments. The hydrogel protrusion set may include balloon 420 positioned between pusher 410 and hydrogel 430. Balloon 420 is used to occlude temporarily the blood vessel when inflated 440 allowing the hydrogel to swell with no blood flow around it until the hydrogel adheres to the vessel wall. Balloon 420 may deform the vessel wall shape and allow the hydrogel to expand into the deformed shape in order to increase the adhesion to the vessel wall.

Optionally, the hydrogel may have a conical shape in its shrunken state configured to swell into a swollen conical shape as shown in FIG. 4 450 in order to increase the adhesion to the vessel wall.

After the hydrogel swells 450 the balloon is deflated 460 and is ready to be removed through sheath 470.

FIG. 5A illustrates a hydrogel protrusion set that includes electro-activating electrodes, according to certain embodiments. Hydrogel protrusion set 500 may include two or more electrodes used to electro-activate the hydrogel. The two or more electrodes may be a central electrode 510 mounted in the hydrogel central hole and an outer electrode 520 mounted on the catheter outer wall 530.

Optionally, more than one electrode may be mounted on the catheter outer wall 530. Optionally, the two or more electrodes may be mounted on the catheter wall 530 circulating the hydrogel without a central electrode.

Optionally, a stimulator unit (not shown) is provided with the hydrogel occlusion system. The stimulator may be configured to deliver programmable electrical stimulation waveforms, wherein the stimulation waveforms may include a DC component, a monophasic or biphasic pulse component, and wherein the stimulation waveforms magnitude may be less than 10 Volts and less than 10 KHZ for patient safety if stimulation is applied internally during deployment. Optionally, the hydrogel may be stimulated externally before deployment allowing applying high voltages (above 10 Volts) and high frequency AC signals (above 10 KHZ) onto the hydrogel. The electrical stimulation is arranged to maintain or change the state of expansion of the hydrogel.

FIG. 5B illustrates an interdigitated electrode configuration, according to certain embodiments. The delivery tool catheter 550 distal end 560 may include a plurality of interdigitated electrodes 570 used to uniformly shrink the hydrogel either externally before deployment or internally during the delivery of EAH 580 to the target occlusion location in its shrunken state. Optionally, the interdigitated electrodes 570 are positioned on the perimeter of EAH 580. Optionally, the interdigitated electrodes are arranged to uniformly shrink EAH 580, either internally in the patient body during deployment or externally before deployment.

Optionally, the hydrogel does not have a central hole and the delivery tool includes other means for holding and detaching the hydrogel in the target occlusion location.

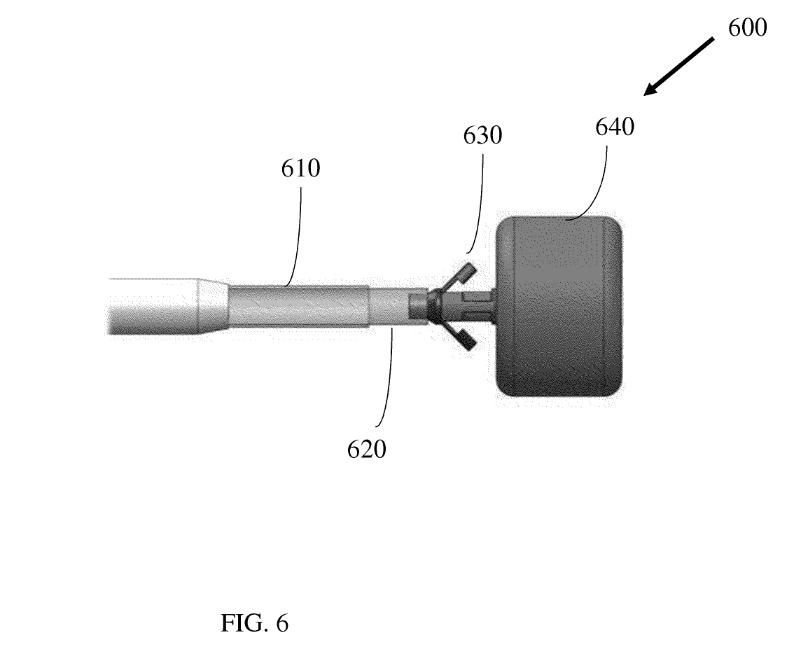

FIG. 6 illustrates a hydrogel protrusion set that includes a pusher with teeth, according to certain embodiments. Hydrogel protrusion set 600 may include a catheter 610 a pusher 620 with teeth at its distal end 630 and a hydrogel 640. The pusher teeth 630 are in a closed position, fixating the hydrogel to the delivery tool, as long as they are covered with the catheter 610. Once the catheter is pulled back the pusher teeth open and the hydrogel 640 is released. With the hydrogel protrusion set that includes pusher teeth 630, there is no need to provide a hydrogel with a central hole for the guidewire. Alternative mechanisms, equivalent to the pusher teeth mechanism described herein, for holding and detaching the hydrogel from the delivery tool may be utilized without exceeding the scope of the present invention.

### Pluronic Methacrylic Acid Sodium Salt Hydrogel (PLMANa) Synthesis

PLMANa hydrogel is synthesized by free radical polymerization. In one embodiment, a formulation is provided for transforming a known “non smart” amphiphilic hydrogel, PF127 bismethacrylate (PF127-BMA) hydrogel into a “smart” and mechanically strengthened electroactive hydrogel, by cross linking the polymer, with the aid of an electrically responsive anionic methacrylic acid sodium salt monomer. PF127 is known to be biocompatible, non toxic and is FDA (Food & Drug Administration) approved in a wide array of molecular weights.

Optionally, 8-15% of Pluronic PF-127-BMA (PL), 24-25% of hydrolyzed methacrylic acid sodium salt (MANa) and 46-52% of demineralized and deionized water (such as MQ, i.e. water filtered by a Milli-Q filter system commercially available from Millipore Corporation of Billerica, Mass., exhibiting a resistivity higher than 18 MΩ*cm), flushed with nitrogen gas for about 30 minutes, are mixed together in a plastic container. The obtained paste is cooled on ice, to prevent physical gelation of Pluronic, mixed again and placed in the refrigerator for about 8 hours. Ammonium persulfate, optionally 0.75 ml of a 1 Molar (1 M) solution, is added as a radical initiator and N,N,N′,N′-tetramethylethylenediamine, optionally 0.75 ml of a 1 Molar solution, as an accelerator. The monomer solution is slowly mixed, to avoid oxygen trapping, and transferred to a refrigerator and thereafter to a water bath set at about 37° C.

Optionally, when the polymerization is complete the hydrogel is removed from the container and washed in an excess of demineralised water to remove any residual material. The hydrogel is then placed in MQ, replaced twice a day, for a period of 4-5 days.

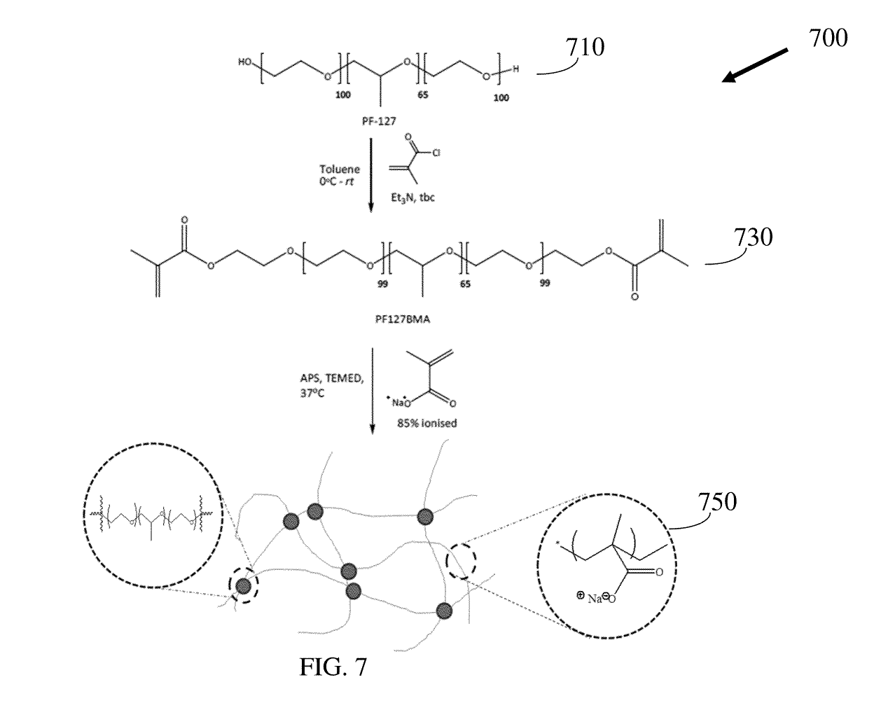

FIG. 7 illustrates the synthesis of a PLMANa hydrogel 750. PF127 polymer 710, which is an ABA block type (co) polymer, that was chosen as the base polymer for synthesizing the PLMANa hydrogel, is first reacted with bismethacrylate-co-methacrylic acid forming the Pluronic PF-127-BMA 730. PF127 base polymer 710 is an amphiphilic poly(ethylene oxide)-b-poly(propylene oxide)-b-(polyethylene oxide) [PEO-PPO-PEO] triblock (co)polymer.

The Pluronic PF-127-BMA 730 is further reacted with hydrolyzed methacrylic acid sodium salt (MANa) and the radical initiator ammonium persulfate (APS) and TEMED used to generate free sulphate (SO₄⁻) radical anions which propagated the conjugation of methacrylate end groups via addition mechanism to form the cross linked polymer PLMANa hydrogel 750.

Uptake and retention of fluid in the cross linked polymer PLMANa hydrogel defines its underlining properties. Upon fluid imbibition, the distance between cross-linked junctions (mesh size) is increased due to hydration of the hydrophilic moieties via occupation of the bound and free volume. This in turn invokes volumetric and mass increase as more fluid is retained. However, a balance between crosslink density and polymer osmotic pressure (due to anions) regulates the degree of swelling. As a consequence, tuning (via controlled swelling) of viscoelastic properties and solute release kinetics can be done to suit varied applications such as the embodiments of present invention hydrogel occlusion systems for example.

The PLMANa hydrogel synthesis steps of the present invention described herein have been adapted to produce a strengthened, un-brittle hydrogel swollen state that reaches an equilibrium swelling in blood environment. The PLMANa hydrogel is transparent and may have a substantially cylindrical shape, 20 millimeters (mm) long with a 3 mm diameter in its shrunken state. The transparent PLMANa hydrogel may reach a diameter of about 6 mm in its swollen state in equilibrium in fluid contact with blood for example. Other shrunken state diameters and lengths and swollen state equilibrium diameters and lengths may be designed according to the required occlusion target sizes.

The concentration of the mobile ions inside the hydrogel, that neutralize MANa, is not equal to that in the outer solution which creates an osmotic pressure difference. Those mobile ions tend to reduce the concentration gradient trying to diffuse to the outer solution. However, electro-neutrality condition prevents them from leaving the hydrogel and the osmotic pressure difference instead pumps water in, causing swelling of the hydrogel. The hydrogel can increase its own weight in MQ up to 43 times before reaching the equilibrium swelling.

The time associated with the hydrogel's swelling process is measured in minutes. As the hydrogel volume increases, the density of charged groups is reduced, thereby, also decreasing the osmotic pressure difference. Each time the water is replaced, the hydrogel reaches its final equilibrium volume as a balance between polymer matrix-solvent affinity, network elasticity which resists expansion, and charged groups-mobile ions interactions.

The PLMANa hydrogel synthesis steps comprise, in one embodiment: (a) obtaining de-ionized distilled water and flushing with nitrogen or argon gas for about 30 minutes; (b) dissolving about 7-14 weight %, optionally 7.2-14.2%, of pluronic-BMA powder (PF127) in methacrylic acid about 22-24%, optionally 21.8-23.8%, weight % liquid, the percentages being of an overall solution; (c) adding about 30-44 weight %, optionally 30-43.8%, de-ionized distilled water to pluronic-BMA solution; (d) preparing 1-10 Molar ammonium persulphate (APS) solution of the de-ionized distilled water flushed in nitrogen or argon gas; (e) preparing 1-10 Molar of TEMED solution of the de-ionized distilled water flushed in argon gas; (f) mixing at room temperature pluronic-BMA, APS and TEMED solutions forming an EAH solution and adding about 4.7-9 weight % porogens generating porosity in the EAH, the percentage being a percentage of the overall EAH solution; (g) cooling the EAH solution on ice for several hours; (h) stirring the cooled EAH solution for about 1 minute; (i) cooling the stirred EAH solution for 8-12 hours in a refrigerator at 4 degrees Celsius; (j) adding 6.8-7.14 weight % APS and 6.8-7.14 weight % TEMED to the EAH solution, the percentages being percentages of the overall EAH solution; (k) pouring the cooled EAH mixture into a mould and leaving the EAH solution to settle at about 4 degrees Celsius in the mould; (1) curing the EAH mixture in a 37° C. water bath for about 3 hours; and (m) removing the porogens by a leaching out method using excessive solvent ensuring no residual porogen is present in the final EAH.

Porogens are added to create a porous hydrogel in order to speed up swelling rate and to create a surface texture that increases adhesion to the vessel wall.

Optionally, beads and/or fibers from biopolymers or other materials that can be subsequently removed or washed away, may be added to the hydrogel to increase its porosity and its swelling rate in fluid contact with blood to its equilibrium swollen state.

Optionally, other hydrogels, other basic polymers, other acid salts, other polymerization schemes and catalysis, and other reaction conditions and steps may be used to synthesize hydrogels, without exceeding the scope of the present invention.

Optionally, the synthesis ingredients and method steps described hereinabove, including the amount of polymer cross linking for example, are adjusted such that the hydrogel characteristics in its swollen state in equilibrium in fluid contact with blood will meet the required characteristics of stability and strength for permanently occluding the target occlusion location.

### Hydrogel Based Occlusion Method

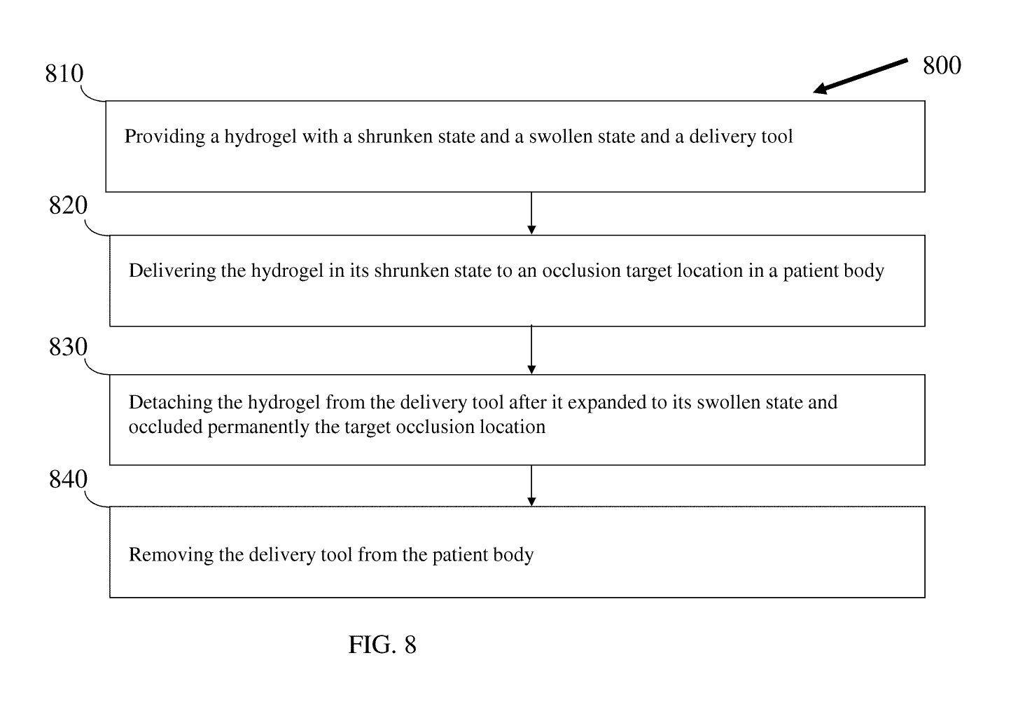

FIG. 8 illustrates a method 800 of occlusion based on hydrogel, according to certain embodiments. The occlusion method 800 includes: in stage 810, providing a hydrogel exhibiting a shrunken state and a swollen state and a delivery tool; in stage 820, delivering the hydrogel in its shrunken state to an occlusion target location in a patient body; in stage 830, detaching the hydrogel from the delivery tool when the hydrogel is in the swollen state, thereby permanently occluding the target occlusion location; and in stage 840, removing the delivery tool from the patient body. Typically, the hydrogel swells from its shrunken state to its swollen state in the target location where it is in fluid contact with blood in 10-90 minutes.

Optionally, the occlusion method 800 includes puncturing a central hole along the hydrogel cylindrical axis and delivering the hydrogel to the target occlusion location on a guidewire. Accordingly, the elongated central hole is configured to be filled by swelling of the hydrogel to its swollen state occluding the target location. Optionally, the central hole along the hydrogel cylindrical axis is performed by mechanical drilling of a substantially cylindrical shape hydrogel in its dried shrunken state. Alternatively, the hydrogel may be molded with a central hole using appropriate molding cast having a central rod. Optionally, a molding cast with more than one central rod may be used in order to mold the hydrogel having more than one hole in order to increase swelling speed in the wet swollen state by increasing the hydrogel to fluid contact surfaces.

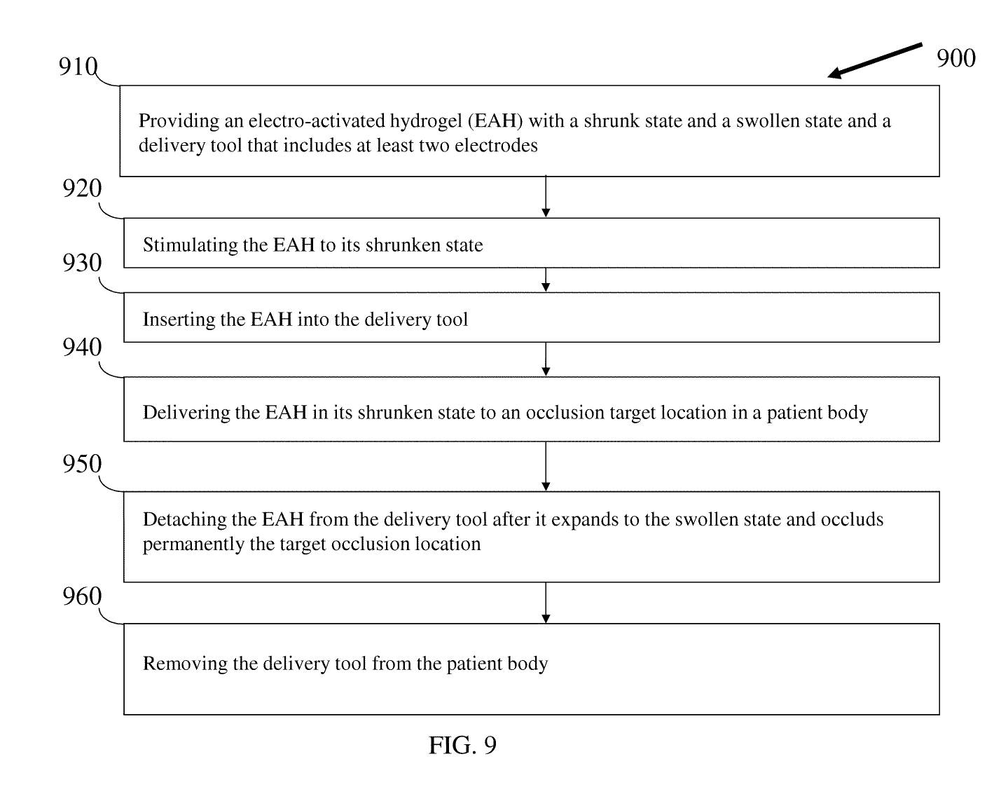

FIG. 9 illustrates the method of occlusion based on electro-activated hydrogel (EAH), according to certain embodiments. The occlusion method 900 includes: in stage 910, providing an EAH exhibiting a shrunken state and a swollen state and a delivery tool that includes at least two electrodes; in stage 920, stimulating the EAH to its shrunken state; in stage 930, inserting the EAH into the delivery tool; in stage 940, delivering the EAH in its shrunken state to an occlusion target location in a patient body; in stage 950, detaching the EAH from the delivery tool after it expands to its swollen state, thereby permanently occluding the target occlusion location; and in stage 960, removing the delivery tool from the patient body.

Optionally, stimulating the EAH to its shrunken state, as described in stage 920, is performed before deployment outside of the patient body. Advantageously, stimulating the hydrogel before deployment allows applying high voltages and high frequency AC signals onto the hydrogel.

Alternatively, stimulating the EAH and keeping it in its shrunken state, as described in stage 920, is performed during deployment of the hydrogel in the patient body through electrodes mounted in the delivery tool distal end.

Optionally, the hydrogel occlusion system stimulator may include impedance sensing circuit for monitoring the EAH swelling rate. Accordingly, a control unit may be attached to the hydrogel protrusion set, wherein the control unit may include a stimulator, a battery, an impedance swelling rate measurement circuit and a telemetry circuit, and wherein the control unit may be used to control the expansion state of the EAH using the impedance swelling rate circuit as a feedback signal for the stimulating circuit.

Optionally, the hydrogel occlusion method may be used to fill atrial or cerebral aneurysm by filling the lumen surrounding a stentgraft.

### Hydrogel Fixation To The Vessel Wall

Optionally, fixation means to the vessel wall may be added to the hydrogel, such as chemical bonding of biomimetic linking molecules attached to the hydrogel surface, configured to strengthen the adherence to the vessel wall in the hydrogel swollen state. The chemical adhering to the vessel wall may be performed by modification of the hydrogel surface using biomimetic linking molecules that target vascular endothelial receptors. Furthermore, a drug may be incorporated in the interior of the hydrogel in order to enhance ligand-receptor interaction strengthening the chemical adhering between the vessel wall and the hydrogel surface.

According to certain embodiments, the biomimetic linking molecules, typically proteins, may connect to the surface of the hydrogel after it is synthesized. The reaction process may be tuned such that only the hydrogel surface and not the interior of the hydrogel react with the biomimetic linking molecules.

Other molecules and molecular mechanisms may be used to chemically bond the hydrogel surface to the target occlusion location wall receptors and the mechanism described above is merely one non limiting example of such chemical surface modification and ligand-receptor bonding that may be used in embodiments of the present invention.

Thus, the occlusion method may include further the step of adding biomimetic linking molecules to the surface of the hydrogel such that chemical bonding between the hydrogel surface and the target occlusion location wall is achieved when the hydrogel swells to its second state

Advantageously, the above described hydrogel occlusion system may be used to occlude blood vessels, aortic and cerebral aneurisms in a minimal invasive procedure.

Another advantage of the occlusion system described above is that the hydrogel synthesis described above is designed to generate a cross linked hydrogel having a stable swollen state in equilibrium in fluid contact with blood.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While preferred embodiments of the present invention have been shown and described, it should be understood that various alternatives, substitutions, and equivalents can be used, and the present invention should only be limited by the claims and equivalents thereof.

What is claimed is:

1. A hydrogel based occlusion system comprising:
a hydrogel having a shrunken and a swollen state; and
a delivery tool configured to deliver said hydrogel to a target occlusion location, wherein said hydrogel is configured to occlude permanently said target occlusion location in the swollen state.

2. The system according to claim 1, comprising further an external device used to shrink said hydrogel before deployment using said delivery tool.

3. The system according to claim 1, wherein said hydrogel has a solid symmetrical shape around its longitudinal axis, wherein said delivery tool is configured to deliver said hydrogel to said target occlusion location using a mechanical assisted detachment system.

4. The system according to claim 1, wherein said hydrogel is generally cylindrically shaped and has a central hole along its cylindrical axis, wherein said delivery tool is configured to deliver said hydrogel to said target occlusion location using a guide wire configured to thread through said central hole, and wherein said central hole is configured to be filled by said hydrogel in said swollen state after said guide wire is removed, thereby occluding said target location.

5. The system according to claim 1, wherein said hydrogel comprises an electro-activated hydrogel (EAH) and wherein said occlusion system comprises further a stimulator for applying electrical stimulations to said EAH to maintain or change the state of expansion.

6. The system according to claim 1, wherein said hydrogel shrunken state is a substantially dried state and said swollen state is a wet state in equilibrium in fluid contact with blood.

7. The system according to claim 1, wherein said target occlusion location is selected from the group consisting of: a blood vessel wherein the occlusion is performed to block blood flow through the vessel; a hepatic artery wherein said occlusion is performed to stop blood flow to a tumor; aortic aneurysm wherein said occlusion is performed to block aortic aneurysm growth and rupture; and cerebral aneurysm wherein said occlusion is performed to block cerebral aneurysm growth and rupture.

8. The system according to claim 6, wherein said stimulator is configured to apply electrical stimulations to said EAH to bring it to its shrunken state before deployment, wherein said EAH is deployed in said target occlusion location in its shrunken state and wherein said EAH is configured to expand to its swollen state when-electrical stimulation is not applied thereon.

9. The system according to claim 1, wherein said delivery tool comprises an introducer sheath, a dilator, an insertion guide, a pusher, a guide wire and a hydrogel protrusion set, wherein said guide wire is inserted first to said target occlusion location through said introducer sheath, wherein said hydrogel is placed in said hydrogel protrusion set in its shrunken state, wherein said pusher is configured to push said hydrogel protrusion set to said target occlusion location on said guide wire and to push said hydrogel out of said hydrogel protrusion set at the target occlusion location after it expands to said swollen state, thereby occluding said target location.

10. The system according to claim 9, wherein said guide wire comprises further a J shape distal end configured to hold said hydrogel in said target occlusion location until it expands to said swollen state.

11. The system according to claim 9, wherein said hydrogel protrusion set comprises further a balloon, said balloon configured to prevent blood flow around said hydrogel at the target location during expansion of said hydrogel to its swollen state.

12. The system according to claim 9, wherein said hydrogel protrusion set comprises further at least two electrodes configured to apply electrical stimulation to said EAH.

13. The system according to claim 12, wherein a first electrode of said at least two electrodes is a central electrode located in said EAH central hole, and a second electrode is an outer electrode located at said hydrogel protrusion set perimeter.

14. The system according to claim 12, wherein said delivery tool comprises further a plurality of interdigitated electrodes, wherein said plurality of interdigitated electrodes is arranged to uniformly shrink the hydrogel either externally before deployment or internally in the patient body.

15. The system according to claim 1, wherein said hydrogel includes biomimetic linking molecules that target vascular endothelial receptors, and wherein a drug is incorporated in the interior of the hydrogel in order to enhance ligand-receptor interaction strengthening chemical adhering between said hydrogel and said vessel wall.

16. An occlusion method, the method comprising:
(a) providing a hydrogel with a shrunken state and a swollen state and a delivery tool;
(b) delivering said hydrogel in its shrunken state to an occlusion target location in a patient body;
(c) detaching said hydrogel from said delivery tool after it expands to its swollen state, thereby permanently occluding the target occlusion location; and
(d) removing the delivery tool from the patient body.

17. The occlusion method of claim 16, wherein said provided hydrogel is an electro-activated hydrogel (EAH), and wherein said method further comprises inserting said EAH into said delivery tool and stimulating said EAH to a shrunken state either externally before deployment or internally in the patient body.

18. The occlusion method of claim 17, wherein said stimulating said EAH to a shrunken state is performed before said inserting said EAH into said delivery tool.

19. The occlusion method of claim 17, further comprising during said delivery of said EAH, stimulating said EAH to control the swelling rate of said EAH during said delivering.

20. A hydrogel synthesis method, the method comprising the steps of:
(a) flushing de-ionized distilled water with nitrogen gas for about 30 minutes;
(b) dissolving about 7-14 weight %, per solution of water, of pluronic-BMA powder (PF127) in about 22-24 weight % methacrylic acid liquid;
(c) adding said de-ionized distilled water, about 30-44 weight % to said pluronic-BMA solution;
(d) preparing 1-10 Molar of ammonium persulphate (APS) solution of said de-ionized distilled water flushed in nitrogen gas;
(e) preparing 1-10 Molar of TEMED solution of said de-ionized distilled water flushed in argon gas;
(f) mixing at room temperature said pluronic-BMA, about 6.81-7.14 weight % APS and about 6.81-7.14 weight % TEMED solutions forming an EAH solution and adding Porogens at about 4.7-9 weight % of the overall polymer mixture so as to generate porosity in the final EAH;
(g) cooling the EAH solution on ice for several hours;
(h) stiffing the cooled EAH solution for several minutes;
(i) cooling the stirred EAH solution for 8-12 hours at about 4 degrees Celsius;
(j) adding about 6.8-7,14 weight % APS and about 6.8-7,14 weight % TEMED to the EAH solution;
(k) pouring the cooled EAH mixture into a mold and leaving the EAH solution to settle for about 1 hour at about 4 degrees Celsius in the mold;
(l) curing the EAH mixture in a 37° C. water bath for about 3 hours; and
(m) removing said porogens by a leaching out method using excessive solvent.
